(19) United States
(12) Patent Application Publication
(10) Pub. No.: US 2022/0133134 A1
(43) Pub. Date: May 5, 2022

(54) IMAGING AND PRESSURE SENSING APPARATUS AND PROBES WITH A SLIDABLE SHEATH

(71) Applicant: KOTL, LLC, Devens, MA (US)

(72) Inventors: Chenyang Xu, Devens, MA (US); Wei Kang, Somerville, MA (US)

(21) Appl. No.: 17/088,755

(22) Filed: Nov. 4, 2020

(51) Int. Cl. A61B 1/00; A61B 8/00; G01N 29/06; A61B 8/08

(52) U.S. Cl. CPC A61B 1/00142; A61B 8/42; A61B 2562/0247; A61B 8/52; G01N 29/0654

(57) ABSTRACT

The invention relates to a medical sensor system with a probe sheath with a sheath distal end configured for insertion through an insertion opening into a lumen of a patient. A pressure signal channel between the sheath ends contains a pressure sensor at the sheath distal end configured to measure pressure in the pressure signal channel and produce a corresponding pressure measurement signal. A sheath retraction mechanism portion of the probe sheath has: i. an extended sheath configuration wherein the sheath distal end extends through the insertion opening into the lumen and encloses the pressure measurement sensor in physical isolation from the lumen, and ii. a retracted sheath configuration wherein the sheath distal end is longitudinally retracted back from the lumen towards the insertion opening so as to expose at least a portion of the pressure measurement sensor to the lumen.

### TECHNICAL FIELD

The present invention relates to a combined intravascular imaging and pressure sensing apparatus and, more particularly, to a combined intravascular ultrasound (IVUS) imaging and intravascular pressure sensing that includes a slidable probe sheath.

### BACKGROUND OF THE INVENTION

During the treatment of stenosis in a blood vessel, lesion severity can be assessed by means of structural imaging and/or measurement of blood pressure. Imaging technologies such as Intravascular Ultrasound (IVUS) and Optical Coherence Tomography (OCT) are utilized for visualization of the blood vessel microstructure. These technologies are used for determining vessel lumen size, stent deployment and other clinically relevant information. In order to acquire localized information of the intraluminal structure, an elongated imaging probe with distal miniature ultrasound or optics assembly is often used. The probe can be configured to deliver some form of probing energy to the tissue and collect a measurement signal to form images.

Functional parameters, such as blood pressure, can also be used to ascertain blood flow and obstruction in blood vessels. Fractional Flow Reserve (FFR), derived from blood pressure sensing, is one well-accepted measurement method to evaluate narrowed or stenotic lesion severity in situ. FFR is defined as the pressure measured distally to the lesion divided by the ostium pressure. This distal measurement utilizes a fine-wire or a probe with a pressure sensor mounted near the distal tip, which can be inserted into the vessel lumen. However, the introduction of the pressure sensor causes partial occlusion of the blood vessel. This occlusion causes more pressure drop in the stenosis due to smaller luminal profile, which in turn causes errors in the FFR measurement.

Acquiring both the structural and pressure information is desirable. The structural images may also improve the FFR measurement accuracy because the blood vessel geometry can be taken into account to compensate for the effect of the partial occlusion caused by the pressure wire or probe. There have been efforts to combine imaging and FFR measurement into a single device, which allows for the acquisition of multimodality information with just one probe insertion. One straightforward approach is to place the imaging channel and the FFR channel side-by-side. However, compared to a single modality probe, this side-by-side arrangement results in an undesirable larger crossing profile. If the combined probe is inserted through the stenosis to measure distal pressure, it will occlude an even larger portion of the lumen, which may further affect the accuracy of FFR measurement.

To achieve a smaller crossing profile, some prior art systems have combined OCT/FFR systems. A compact combined system uses a single optical fiber core for both the OCT and FFR probes. However, because OCT involves coherent imaging, OCT typically requires a single-mode fiber. Unfortunately, single-mode fiber has a very small core diameter and often a small numerical aperture. Consequently, light reflected from only a small portion of the FFR pressure transducer can be collected by the single-mode fiber, which leads to a low signal-to-noise ratio (SNR) and requires a very meticulous, and therefore expensive, alignment process during probe manufacture. There exists a need for a cost-effective solution to achieve a smaller crossing profile in the combined imaging/FFR measurement probe.

U.S. Pat. Publ. No. 2014/0094697 by Christopher Petroff, et al. (“Petroff”, incorporated herein by reference in its entirety), describes existing equipment and methods for treating blood vessels with stenotic lesions and other full or partial blockages. U.S. Pat. No. 8,478,384 to Joseph M. Schmitt, et al. (“Schmitt”, incorporated herein by reference in its entirety), describes a combined OCT/pressure measurement probe and provides basic information about OCT.

### SUMMARY OF THE INVENTION

Embodiments of the present invention are directed to a medical sensor system with a probe design for combined intravascular imaging and pressure sensing. There is a probe sheath that has a sheath proximal end and a sheath distal end. The sheath distal end is configured for insertion through an insertion opening into a lumen of a patient. There is a pressure signal channel between the sheath ends. A pressure sensor is coupled to the pressure signal channel at the sheath distal end and is configured to measure pressure in the pressure signal channel and produce a corresponding pressure measurement signal. A sheath retraction mechanism portion of the probe sheath has: i. an extended sheath configuration wherein the sheath distal end extends through the insertion opening into the lumen and encloses the pressure measurement sensor in physical isolation from the lumen, and ii. a retracted sheath configuration wherein the sheath distal end is longitudinally retracted back from the lumen towards the insertion opening so as to expose at least a portion of the pressure measurement sensor to the lumen.

In further specific embodiments, the probe sheath further includes an exit opening at the sheath distal end having an opening diameter substantially larger than a cross-sectional diameter of the pressure sensor. The probe sheath may further include an imaging signal channel between the sheath ends adjacent the pressure signal channel, and an imaging sensor coupled to the imaging signal channel at the sheath distal end and configured to produce a corresponding imaging measurement signal.

The system also may include a rotatable torque arrangement within the probe around at least a portion of both the signal channels and configured to rotate the sensors within the sheath distal end. The rotatable torque arrangement may include a torque coil, a flexible tubing, and/or a micro-motor within the sheath distal end. A micro-motor may be configured to rotate the imaging sensor, or a reflector that reflects imaging energy from the imaging sensor.

In another specific embodiment, the system also comprises a rotatable torque arrangement within the probe around at least a portion of the imaging signal channel and configured to rotate the imaging sensor within the sheath distal end. In such an embodiment, the probe sheath may have a first bore for the imaging signal channel and a second bore for the pressure signal channel such that the two channels are in physical isolation.

In specific embodiments, the pressure sensor may be an electrical pressure sensor or an optical pressure sensor.

The system may be an intravascular ultrasound (IVUS) imaging system, an intravascular Optical Coherence Tomography (OCT) imaging system, or a spectroscopy imaging system.

### BRIEF DESCRIPTION OF THE DRAWINGS

FIG. 1 provides an overall view of a combined imaging and pressure sensing probe according to one embodiment of the present invention.

FIG. 2 provides a side cross-sectional view of the distal end of the probe sheath according to one embodiment of the present invention.

FIG. 3 depicts a specific operation mode where the pressure distal to a stenosis is to be acquired.

FIG. 4 shows a cross-sectional end view of a specific embodiment of the probe sheath.

FIG. 5 shows a side cross-sectional view of the distal end of the probe sheath according to another embodiment of the present invention.

FIG. 6 illustrates a side cross-sectional view of a specific embodiment of a proximal mating unit.

FIG. 7 illustrates a side cross-sectional view of yet another specific embodiment of the distal end of the probe which has a micro-motor.

FIG. 8 illustrates a side cross-sectional view of yet another specific embodiment of the distal end of the probe.

FIG. 9 illustrates the cross-sectional view of another specific embodiment of the probe body.

FIG. 10 shows an end cross-sectional view of another specific embodiment of the proximal mating unit.

FIG. 11 illustrates a specific embodiment of a medical sensor system that can be used with the combination probe.

### DETAIL DESCRIPTION

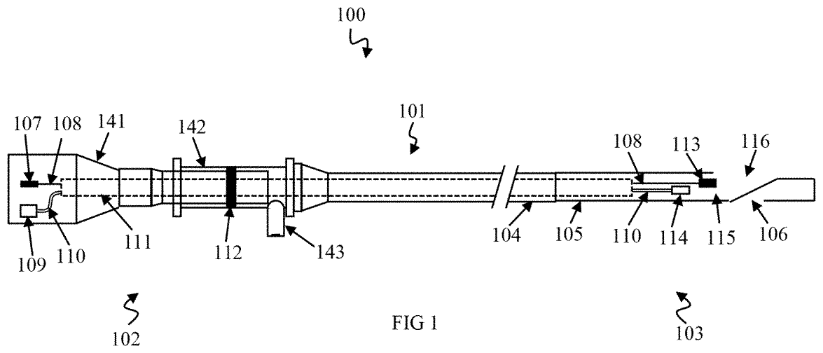

The following description refers to the accompanying drawings that illustrate certain embodiments of the invention in more detail. FIG. 1 provides an overall view of a specific imaging and pressure sensing combination probe 100 according to one specific embodiment of the present invention in the specific form of a catheter for easy insertion into a lumen of an imaging patient such as a blood vessel. The probe 100 includes a probe sheath 101 with a sheath proximal end 102 and a sheath distal end 103 where a proximal end outer boundary 104 transitions to a distal end inner boundary 105. The probe sheath 101 can be made of materials with substantially little attenuation in the mechanical frequency band used for ultrasound imaging.

The sheath proximal end 102 here includes a pressure connector 109, an imaging connector 107, a proximal mating unit 141, a telescoping section 142 and a liquid purge port 143. The imaging connector or the pressure connector can be either optical connector or electrical connector. The sheath distal end 103 includes a rapid-exchange section 106, an imaging sensor 113, for example, an ultrasonic transducer or an OCT transducer, and a pressure sensor 114. An imaging sensor 113 in the specific form of an ultrasound transducer is configured to transmit ultrasound energy and acquire image information about the luminal structure. The pressure sensor 114 is configured to sense ambient pressure within the lumen and be an optical pressure sensor or an electrical sensor. A signal transmission line 108 connects the imaging sensor 113 and the imaging connector 107, forming an imaging signal channel. A signal transmission line 110 connects the pressure sensor 114 and the pressure connector 109, forming a pressure signal channel. The signal transmission line is either an optical fiber or an electrical transmission line, depending on the working mechanism of the imaging sensor or the pressure sensor.

Within the probe sheath 101 there also is an inner bore 115 that contains rotary inner parts including an electrical lead 108, an optical fiber 110, a rotatable torque arrangement 111 and an O-ring seal 112. The rotatable torque arrangement 111 lies within the probe sheath 101 around at least a portion of the signal channels at the sheath proximal end 102 and is configured to rotate the sensors 113 and 114 at the sheath distal end 103.

The inner bore 115 as shown has at least one bore opening 116 exposed to the environment of the tissue lumen. When the sheath distal end 103 is retracted with respect to the imaging sensor 113 and the pressure sensor 114, the two sensors can be exposed from the bore opening 116 into the tissue lumen. The exit dimension of the opening 116 is substantially larger than the cross-profile of the sensor assembly 113 and 114. When the sheath distal end 103 is extended with respect to the two sensors, the sheath distal end 103 can fully encompass the two sensors so that they can be rotated.

Without loss of generality, the embodiments below are explained with the configuration where an optical pressure sensor with optical fiber, an electrical imaging transducer with electrical transmission line and a torque coil rotational arrangement are used. Other configurations are similar and are also within the scope of this intervention.

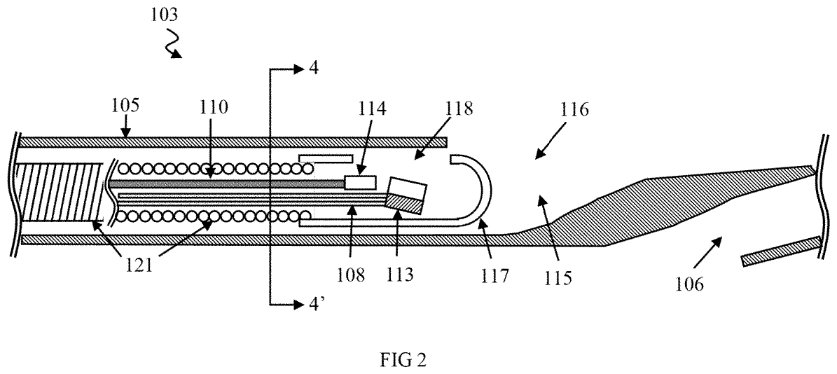

FIG. 2 provides a side cross-sectional view of the distal end 103 of the probe sheath 101 according to one embodiment of the present invention. A torque coil 121 lies within the probe sheath 101 around at least a portion of the signal channels—the optical fiber 110 and electrical wires that form the electrical lead 108—and is configured to rotate the sensors 113 and 114 within the sheath distal end 103. For example, the imaging sensor 113 here is ultrasound transducer that is oriented to transmit ultrasound energy generally radially relative to the longitudinal axis of the torque coil 121. The optical fiber 110 is so arranged that it is not in the energy transmission path of the ultrasound transducer imaging sensor 113. The torque coil 121 can be single layered or multiple layered.

In the embodiment in FIG. 2, there is an additional protective housing 117 that is attached to the torque coil 121 to encompass the imaging sensor 113 and the pressure sensor 114. This protective housing 117 can be configured as shown to form a round distal tip. The protective housing 117 here has a side opening 118 such that the wall of the protective housing 117 does not affect the transmission of ultrasound energy from the imaging sensor 113. The side opening 118 also allows the pressure outside the protective housing 117 to be transmitted to the pressure sensor 114. The protective housing 117 can be made of metal or polymer, which can be glued or welded to the torque coil 121. In some embodiments, there can be another opening at the distal tip of the protective housing 117 that is configured to further facilitate the purge of air bubbles that otherwise can be trapped inside the protective housing 117 during use.

In the embodiment depicted in FIG. 2, the sheath distal end 103 of the combination probe 100 can be inserted into the tissue lumen by using the rapid exchange section 106 in conjunction with a guidewire. The imaging sensor 113 and the pressure sensor 114 then can be rotated within the inner bore 115 and both structural and pressure information can then be acquired as needed.

Thus the probe sheath 101 has a sheath retraction functionality with two modes: (1). an extended sheath configuration wherein the sheath distal end 103 extends through the insertion opening into the lumen and encloses the sensors 113 and 114 so that they can be rotated by the rotatable torque arrangement 111 in physical isolation from the lumen, and (2) a retracted sheath configuration wherein the sheath distal end 103 is longitudinally retracted back from the lumen towards the insertion opening so as to expose at least a portion of at least one of the sensors 113 and 114 to the lumen for performing sensor measurements.

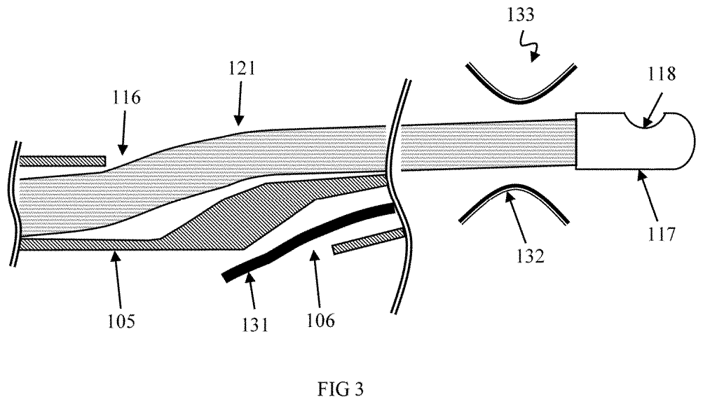

FIG. 3 illustrates another specific operation mode where the pressure distal to a stenosis 132 in a blood vessel lumen 133 is acquired. The sheath distal end 103 can first be inserted through the lesion such that the pressure sensor assembly 114 encompassed by the protective housing 117, is distal to the stenosis 132. The torque coil 121 is then locked to stay relatively stationary with respect to the blood vessel lumen 133, while the distal end inner boundary 105 of the probe sheath 101 is retracted along the guidewire 131 in the rapid exchange 106 until its entirety becomes proximal to the stenosis 132. In this mode, the crossing profile of the probe at the stenosis 132 is smaller than the size of the distal end inner boundary 105 of the probe sheath 101. The pressure measured by the pressure sensor 114 may be more accurate.

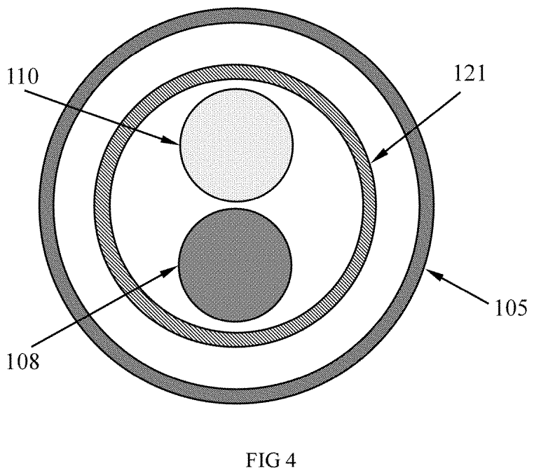

FIG. 4 shows a cross-sectional end view of the specific embodiment shown in FIG. 2 along the line 4-4′. The torque coil 121 encompasses both the electrical lead 108 and the optical fiber 110. The torque coil 121, the electrical lead 108 and the optical fiber 110 can be rotated as one unit inside the distal end inner boundary 105 of the probe sheath 101. The torque coil 121 can be single layered or multiple layered.

This foregoing are specific embodiments of combining ultrasound imaging and optical pressure measurement into one probe. The combination of these two technologies merely illustrates the principles of the present invention. Various modifications and alterations to the described embodiments can be apparent to those skilled in the art in view of the teaching herein. Indeed, the imaging modality used in the described specific embodiments can also be configured to be an optical imaging technology, such as but not limited to Optical Coherence Tomography (OCT) or spectroscopy. On the other hand, the pressure sensing technology can be configured to use an electrical pressure sensor.

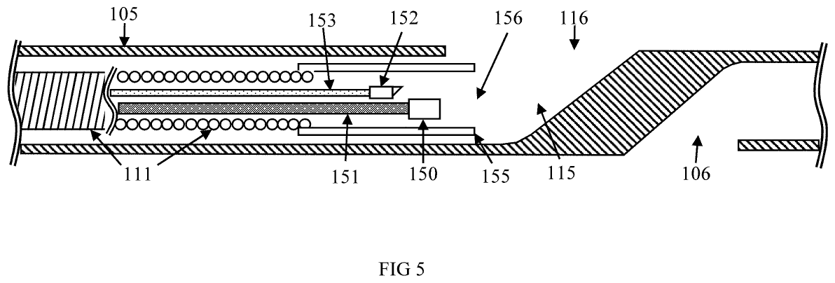

A specific embodiment which combines OCT imaging and an electrical pressure sensor is shown in FIG. 5. The electrical pressure sensor 150 is in communication with electrical wires that form an electric lead 151. An OCT imaging optical lens sensor 152 is in communication with an optical fiber 153. The electrical lead 151 is so arranged that it is not in the energy transmission path of the OCT optical lens sensor 152. A protective housing 155 can be made of material of substantially small attenuation in the optical frequency band used for the OCT imaging. A distal opening 156 can facilitate purging air bubbles from the protective housing 155 and allows the pressure outside the housing to be transmitted to the pressure sensor 150.

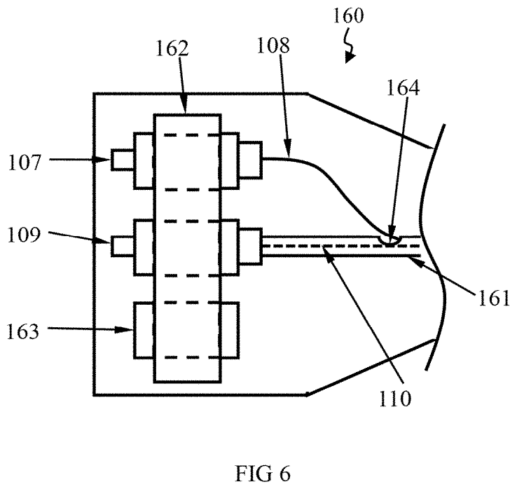

FIG. 6 illustrates a specific embodiment of the proximal mating unit 124 associated with the specific arrangements shown in FIGS. 2 and 4. The mating unit 124 includes an optical fiber tube 161 which can be the same hypodermic tubing. It can also be some other hollow flexible tube. The optical fiber 110 for pressure sensing can be positioned inside the optical fiber tube 161 and be terminated by an optical connector 109. The optical connector 109 can be attached to the optical fiber tube 161. In another specific embodiment, there can be a small tube opening 164 on the optical fiber tube 161 which allows for the exit of the electrical lead 108 for ultrasound imaging. The electrical lead 108 is terminated by an electrical connector 107. There can be a mating arrangement 162 such as a frame which attaches the two connectors 107 and 109 such that they can be rotated as one unit for ultrasound imaging. There can also be one or more counterbalancing weights 163 to balance the centrifugal forces during rotation.

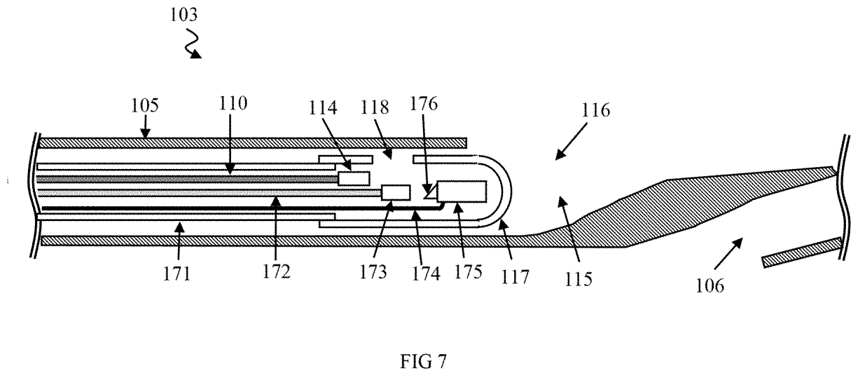

FIG. 7 illustrates the distal end of another specific embodiment of the present invention which comprises a micro-motor 175. An optical lens assembly 173 is utilized here to illustrate the principles of the present invention. The stator of the micro-motor 175 is fixated to the protective housing 117. An optical mirror 176 is fixated to the rotor of the micro-motor 175. The wires 174 provide electrical power to the micro-motor 175. The optical lens assembly 173 attached to the distal end of the optical fiber 172 is so configured that the optical mirror 176 is at the optical axis of the lens assembly 173 and reflects the optical beam generally radially relative to the optical axis. Optical scanning can be achieved by rotating the mirror 176. The signal channels 110 and 172 and the power wires 174 are enclosed with a longitudinal tubing 171 which can be made of metal or polymer.

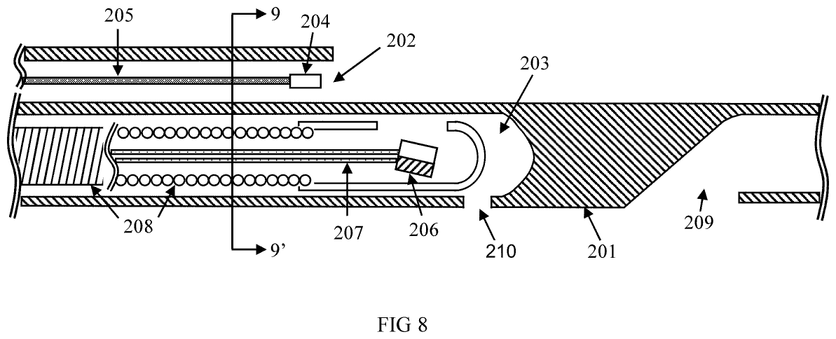

This foregoing are specific embodiments where imaging and pressure signal channels are configured in one single bore of the probe sheath. In these configurations, the imaging sensor and the pressure sensor can be rotated as a single unit. However, if the two signal channels are separated in within separate individual bores, it is not necessary to rotate the pressure sensor. FIGS. 8-10 depict a specific embodiment of such an arrangement.

Referring to FIG. 8, probe sheath 201 has a first bore 202, a second bore 203, and a rapid exchange 209. The first bore 202 contains an optical pressure sensor 204 in communication with optical fiber 205. The second bore 203 contains an ultrasound imaging transducer 206 in communication with electrical wires 207 and a torque coil 208. The torque coil 208, the electrical wires 207 and the ultrasound imaging transducer 206 can be rotated as one unit. The second bore 203 can have at least one bore opening 210 to the lumen environment for air bubble purging. When the ultrasound imaging is performed, pressure sensor 204 is maintained proximal to the ultrasound imaging transducer 206 so that it does not interfere with the ultrasound energy transmission path. In some embodiments, the probe sheath 201, the ultrasound imaging transducer 206 and the torque coil 208 can be configured to be retractable with respect to the tissue lumen while maintaining the pressure sensor 204 stationary with respect to the tissue lumen. When a pressure measurement is performed, the pressure sensor 204 can be exposed to a stenosis in the tissue lumen distal to the rapid exchange 209.

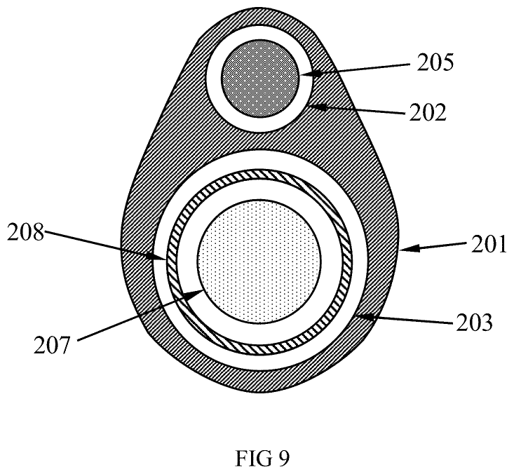

FIG. 9 shows a cross-sectional view of the embodiment in FIG. 8 cut along the line 9-9′. The probe sheath 201 include the first bore 202 and the second bore 203. In the second bore 203, the torque coil 208 surrounds the electrical wires 207 and can be rotated. The optical fiber 205 is in the first bore 202. The cross-section of the probe sheath 201 can be designed in arbitrary shape, preferably with a small crossing profile.

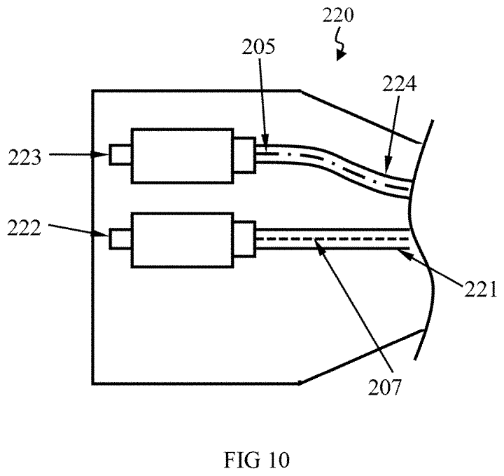

FIG. 10 illustrates a specific embodiment of the proximal mating unit 220 associated with the specific embodiment presented in FIGS. 8 and 9. The mating unit 220 includes a torque tube 221 extended along a longitudinal direction. The torque tube 221 can be a hypodermic tubing or some other hollow flexible tube. The electrical wire 207 is extended within the inner lumen of the torque tube 221 and is terminated with an electrical connector 222. The torque tube 221 can be attached to the torque coil 208 such that the electrical connector 222, the torque tube 221, the electrical wire 207 and the torque coil 208 can be rotated as one unit. The optical fiber 205 is terminated with the optical connector 223. A supporting sheath 224 near the optical connector 223 provides compressive strength when the probe sheath 201 is retracted. Longitudinal movement of the electrical connector 222 and the optical connector 223 can be controlled independently or in coordination. During retraction of the probe sheath 201, the electrical connector 222 is pulled back sufficient distance, which in turned pulls back the ultrasound imaging transducer 206 to avoid jamming the torque coil 208 within the probe sheath 201. The optical connector 223 and its supporting sheath 224 are maintained stationary during the retraction of the probe sheath 201 or the retraction of the ultrasound imaging transducer 206, which allows the optical pressure sensor 204 to be exposed outside the probe sheath 201.

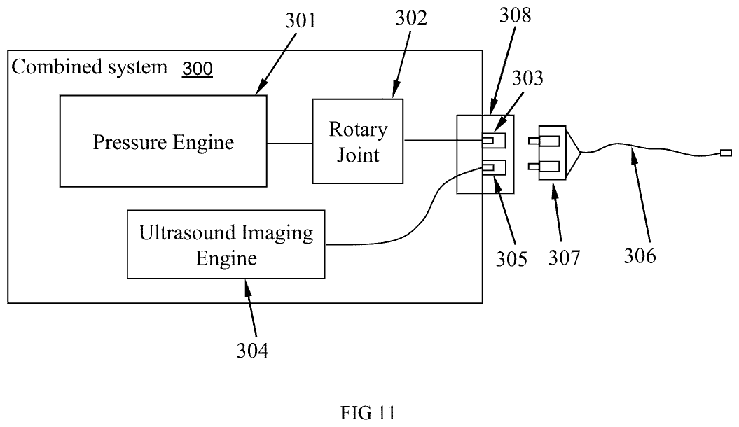

FIG. 11 illustrates a specific embodiment of a combined ultrasound imaging/pressure measurement system 300 which can be used in conjunction with the combination probe described in FIGS. 1-7. The measurement system 300 includes a pressure engine 301, an optical rotary joint 302, a pressure sensing mating sleeve 303, an ultrasound imaging engine 304 and an ultrasound imaging mating sleeve 305. The combined ultrasound imaging/pressure measurement probe 306 can be mated via the proximal mating unit 307. A rotation driver unit 308 can provide the torque needed for ultrasound imaging.

1. A medical sensor system comprising:
a probe sheath having:
i. a sheath proximal end and a sheath distal end, the sheath distal end being configured for insertion through an insertion opening into a lumen of a patient,
ii. a pressure signal channel between the sheath ends;
a pressure sensor coupled to the pressure signal channel at the sheath distal end and configured to measure pressure in the pressure signal channel and produce a corresponding pressure measurement signal; and
a sheath retraction mechanism portion of the probe sheath having:
i. an extended sheath configuration wherein the sheath distal end extends through the insertion opening into the lumen and encloses the pressure measurement sensor in physical isolation from the lumen, and
ii. a retracted sheath configuration wherein the sheath distal end is longitudinally retracted back from the lumen towards the insertion opening so as to expose at least a portion of the pressure measurement sensor to the lumen.

2. The system of claim 1, wherein the probe sheath further comprises an exit opening at the sheath distal end having an opening diameter substantially larger than a cross-sectional diameter of the pressure sensor.

3. The system of claim 1, wherein the probe sheath further comprises an imaging signal channel between the sheath ends adjacent the pressure signal channel, and an imaging sensor coupled to the imaging signal channel at the sheath distal end and configured to produce a corresponding imaging measurement signal.

4. The system of claim 3, where the system further comprises a rotatable torque arrangement within the probe around at least a portion of both the signal channels and configured to rotate the sensors within the sheath distal end.

5. The system of claim 4, wherein the rotatable torque arrangement includes a torque coil.

6. The system of claim 4, wherein the rotatable torque arrangement includes a flexible tubing.

7. The system of claim 3, wherein the system further comprises a micro-motor within the sheath distal end.

8. The system of claim 7, wherein the micro-motor is configured to rotate the imaging sensor.

9. The system of claim 7, wherein the micro-motor is configured to rotate a reflector that reflects imaging energy from the imaging sensor.

10. The system of claim 3, further comprising a rotatable torque arrangement within the probe around at least a portion of the imaging signal channel and configured to rotate the imaging sensor within the sheath distal end.

11. The system of claim 10, wherein the probe sheath has a first bore for the imaging signal channel and a second bore for the pressure signal channel such that the two channels are in physical isolation.

12. The system of claim 1, wherein the pressure sensor is an electrical pressure sensor.

13. The system of claim 1, wherein the pressure sensor is an optical pressure sensor.

14. The system of claim 1, wherein the system is an intravascular ultrasound (IVUS) imaging system.

15. The system of claim 1, wherein the system is an intravascular Optical Coherence Tomography (OCT) imaging system.

16. The system of claim 1, wherein the system is a spectroscopy imaging system.
